(19) United States
(12) Patent Application Publication
(10) Pub. No.: US 2004/0265179 A1
(43) Pub. Date: Dec. 30, 2004

(54) Pump system and method for transferring hyperpolarized gases

(76) Inventors: Ian Nelson, Chapel Hill, NC (US); Stephen Kadlecek, Carrboro, NC (US); John Nouls, Durham, NC (US)

Correspondence Address: Amersham Health, Inc., IP Department, 101 Carnegie Center, Princeton, NJ, 08540, US

(21) Appl. No.: 10/760,876

(22) Filed: Jan. 20, 2004

Related U.S. Application Data
(60) Provisional application No. 60/440,747, filed on Jan. 17, 2003.
(60) Provisional application No. 60/484,460, filed on Jul. 2, 2003.

(51) Int. Cl. B32B 5/02; B32B 27/12; F01N 3/10; B32B 27/04; G01N 7/00; G01N 31/00; G01N 21/00; G01N 27/00; F04B 17/00; F04B 47/08; G01N 24/00; G01N 33/00; F04B 35/00

(52) U.S. Cl. 422/83; 422/173; 417/375; 436/173

(57) ABSTRACT

A pumping system for hyperpolarized gases employs a reversible fluid flow against a deflectable gas transport bladder. Inflation and deflation of the gas transport bladder is operably associated with valves for directing the flow of the hyperpolarized gas. A second gas transport bladder may be operably associated with additional valving so as to provide more continuous hyperpolarized gas flow. The first and second gas transport bladders may be arranged in-line with a reversible pumping mechanism.

### RELATED APPLICATIONS

The present invention claims priority to U.S. Provisional Patent Application No. 60/440,747, fled Jan. 17, 2003 and also claims priority to U.S. Provisional Patent Application No. 60/484,460, filed Jul. 2, 2003, the entire disclosures of which are all hereby incorporated by reference as if having been fully disclosed herein.

### FIELD OF THE INVENTION

The present invention relates to the field of hyperpolarized gases. More specifically, the present invention is directed to systems and methods for handling hyperpolarized gases.

### BACKGROUND OF THE INVENTION

The use of hyperpolarized noble gases, such as ¹²⁹Xe and ³He, for imaging and spectroscopic applications is becoming more widely applied. Hyperpolarized gases are particularly useful contrast agents for magnetic resonance imaging of body cavities. Once the gas is polarized, each of these applications require some amount of gas handling. It has been seen that the handling and transfer of hyperpolarized gases can have a detrimental effect on the level of polarization. For example, it is known that different materials used to form the gas handling systems have differing effects on the gas polarization. In many handling and transfer systems it is necessary to either compress or expand the as so as to effect the as transfer. It is desirable to achieve the gas transfer without losing polarization, particularly if the polarization is induced at low pressure (˜0.1 kPa) via metastability-exchange optical pumping (MEOP). It is known that the use of commercially available pumps (e.g. diaphragm pumps) leads to unacceptably high polarization losses. There is therefore a need for a system for transferring all of a polarized gas from one container to another while minimizing polarization losses.

### SUMMARY OF THE INVENTION

In view of the needs of the art, the present invention provides a pump system for transferring hyperpolarized gases. The pump system includes an elongate fluid line defining an elongate passageway for transporting hyperpolarized gases therethrough. A first inlet valve and a first outlet valve are spacedly positioned in fluid communication with the fluid line so as to provide interruptible and directable flow therethrough. A first pump chamber defines an interior cavity and includes a collapsible gas bladder defining a gas cavity in fluid communication with the fluid line between the first inlet and outlet valves. The gas bladder further defines a first fluid cavity opposite the bladder from the gas cavity. The system further includes a fluid reservoir defining a second fluid cavity and a pumping mechanism positioned in fluid communication between the first an second fluid cavities. The pumping mechanism is able to direct fluid between the first and second fluid cavities so as to urge the gas bladder between an inflated and deflated configuration.

The pump system of the present invention may further include a second inlet valve and a second outlet valve spacedly positioned on the fluid line so as to provide interruptible and directable flow therethrough. The fluid reservoir then further includes a second collapsible gas bladder defining a second gas cavity in fluid communication with the fluid line between the second inlet and outlet valves. The second gas bladder further fluidly isolates the second gas cavity form the second fluid cavity. The second inlet and outlet valves are operably associated with said first inlet and outlet valves so as to provide for near continuous pumping of a polarized gas.

The present invention further provides a pump for transferring hyperpolarized gas having an elongate first fluid line defining an elongate passwageway for transporting hyperpolarized gases therethrough, and a first pump chamber defining a first interior cavity. The first pump chamber includes a first deflectable gas bladder defining a first gas cavity in fluid communication with the first fluid. The first gas bladder further defines a first fluid cavity opposite the first gas bladder from the first gas cavity. The first gas bladder is deflectable between a first configuration and a second configuration within the first interior cavity so as to draw in and expel a hyperpolarized gas through the first fluid line. An elongate second fluid line defines an elongate passageway for transporting hyperpolarized gases therethrough. A second pump chamber defines a second interior cavity and includes a second deflectable gas bladder defining a second gas cavity in fluid communication with the second fluid line. The second gas bladder further defines a second fluid cavity opposite the second gas bladder from the second gas cavity. The second gas bladder is deflectable between a first configuration and a second configuration within the second interior cavity so as to draw in and expel a hyperpolarized gas through the second fluid line. The pump further includes a fluid transfer device having a housing defining a first drive chamber in fluid communication with the first fluid cavity and a second drive chamber in fluid communication with the second fluid cavity. The fluid transfer device including a fluid drive mechanism for directing fluid between the first fluid cavity and the first dirve chamber and between the second fluid cavity and the second drive chamber so as to respectively cause the first and second bladders to deflect between their first and second configurations.

### BRIEF DESCRIPTION OF THE DRAWINGS

FIG. 1 depicts a first hydraulic pump of the present invention.

FIG. 2 depicts an alternate embodiment of the hydraulic pump of the present invention.

FIG. 3 depicts yet another embodiment of the hydraulic pump of the present invention.

FIG. 4 is a cross-sectional view of the pump of FIG. 3 taken through the line 4-4.

FIG. 5 is an exploded view of the pump chamber of FIGS. 3 and 4.

FIG. 6 depicts the pump chamber of FIGS. 3 and 4 having its diaphragm fully deflected so as to expel hyperpolarized gases from the gas cavity.

FIG. 7 depicts even still another embodiment of the pump of the present invention incorporating two pump chambers of FIGS. 3 and 4.

FIG. 8 depicts the pump of FIG. 7 in a configuration expelling hyperpolarized gas.

FIG. 9 depicts yet still another pump of the present invention incorporating two pump chambers of FIGS. 3 and 4.

FIG. 10 is an alternate depiction of the pump of FIG. 9.

FIG. 11 depicts even yet another pump of the present invention incorporating two pump chambers of FIGS. 3 and 4 and fluid drive mechanism incorporation a dual-headed piston.

FIG. 12 is an alternate depiction of the pump of FIG. 11.

FIGS. 13A-E depict another pump of the present invention.

FIG. 14A-D depict still another pump of the present invention, incorporating a pleated deflectable member.

FIG. 15A-B depict a polarizer incorporating a pump of the present invention

### DETAILED DESCRIPTION OF THE PREFERRED EMBODIMENTS

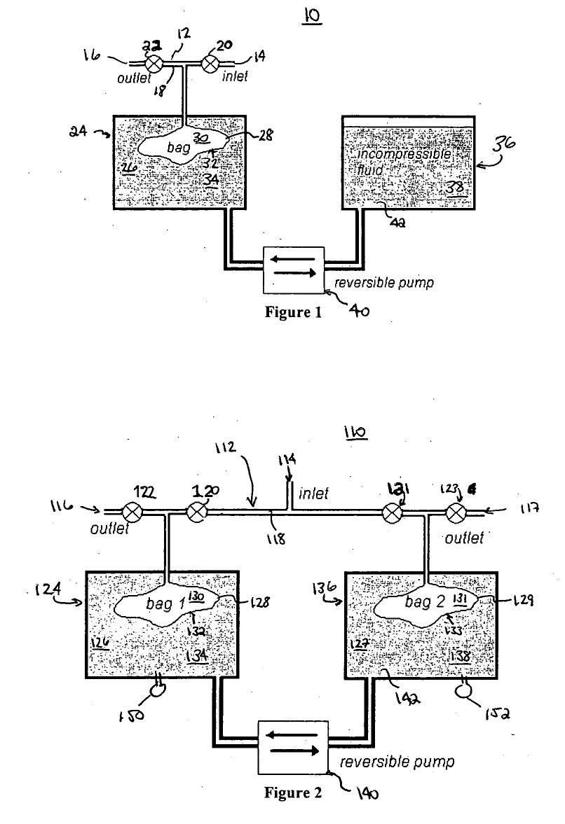

As shown in FIG. 1, the present invention provides a pump system 10 having an elongate hollow fluid line 12 defining an inlet port 14, an outlet port 16 and an elongate passageway 18 extending therebetween for transporting hyperpolarized gases therethrough. First inlet valve 20 and first outlet valve 22 are spacedly positioned within fluid line 12 so as to provide interruptible and directable flow therethrough. System 10 includes a first pump chamber 24 defining an interior cavity 26. A collapsible gas bladder 28 is affixed within cavity 26 so as to define a gas cavity 30 in fluid communication with passageway 18 of fluid line 12 at a location between inlet and outlet valves 20 and 22. Gas bladder 28 includes a bladder wall 32 which further defines cavity 26 to comprise a first fluid cavity 34 exterior to bladder wall 32 and gas cavity 30 interior to bladder wall 32.

Pump system 10 further includes a fluid reservoir 36 defining a second fluid cavity 38. A pumping mechanism 40 is positioned in fluid communication between first and second fluid cavities 34 and 38. Pumping mechanism 40 is able to direct drive fluid 42 between first and second fluid cavities 34 and 38 so as to urge gas bladder 28 between an inflated and deflated configuration. Pumping mechanism 40 is contemplated to be either a reversible pump, or a one-way pump and a system of valves employed to pump the fluid in either direction. Likewise, pumping mechanism 40 may be pressure-compensated, or a bypass valve may be included to allow for deadhead operation.

Pump system 10 desirably employs as drive fluid 42 an incompressible fluid within fluid cavities 34 and 38 and within pumping mechanism 40, although it is also, contemplated that drive fluid 42 may be a compressible fluid or gas as well. Actuation of pumping mechanism 40 causes the transfer of fluid 42 between fluid cavities 34 and 38 so as to cause the sequential or intermittent inflation and deflation of gas bladder 28 within pump housing 24. Inlet and outlet valves 20 and 22 are operably associated with the inflation and deflation of gas bladder 28 so as to direct the flow of hyperpolarized gas from inlet port 12 into gas cavity 30 and through outlet port 14 as will be described further hereinbelow.

Gas bladder 28 is desirably formed from a flexible material so as to allow bladder wall 32 to deflect between the inflated configuration and the deflated configuration and thereby intake and expel any hyperpolarized gas contained into and from gas cavity 30. It is contemplated that gas bladder 28 may be formed from an expandable or elastomeric material. Gas bladder 28 is desirably formed from a material which is selected for having a lower deleterious effect on the polarization of the hyperpolarized gas contained thereby, such as a polymeric material. It is still further contemplated that gas bladder 28 may take the form of an elastomeric diaphram extending across cavity 26. Gas bladder 28 is further desirably formed from a gas-impermeable material so as to prevent leakage of gases which may effect the polarization or the mixture of the polarized gas being pumped.

Polarization losses during the pumping cycle are kept low by virtue of 1) the polymer material can be chosen to be relatively non-depolarizing, and 2) the internal pump volume can be kept relatively large (e.g. 1 liter in our current design), resulting in a favorable surface-to-volume ratio for minimizing surface contact. Initial studies suggest that >95% of the ³He polarization will be retained by gas cycling through pump system 10.

A typical pumping cycle is as follows:

1) Compression: Inlet valve 20 is closed, fluid 42 is pumped from second fluid cavity 38 to first fluid cavity 34, and outlet valve 22 is opened

2) Intake: Outlet valve 22 is closed, the flow direction of fluid 42 is reversed to flow from first fluid cavity 34 to second fluid cavity 28, and inlet valve 20 is opened.

3) repeat

During the compression stage, fluid 42 compresses gas bladder 28, forcing the hyperpolarized gas through outlet valve 14. The highest absolute pressure attainable at the outlet is roughly equal to the differential pressure capability pumping mechanism 40.

During the intake stage, the fluid pressure in the first fluid cavity 38 drops, drawing hyperpolarized gas from inlet port 12 into gas bladder 28. If the fluid level in fluid cavities 34 and 38 is low (such that the level drops below the level of the bag), the lowest pressure attainable in the bag is equal to the vapor pressure of the fluid used. (Then, care must be taken to avoid overinflating gas bladder 28.) If the fluid level is instead chosen such that second fluid cavity 38 is completely filled with fluid before gas bladder 28 is completely inflated, the lowest pressure attainable will be slightly higher, although it will then be impossible to overinflate gas bladder 28.

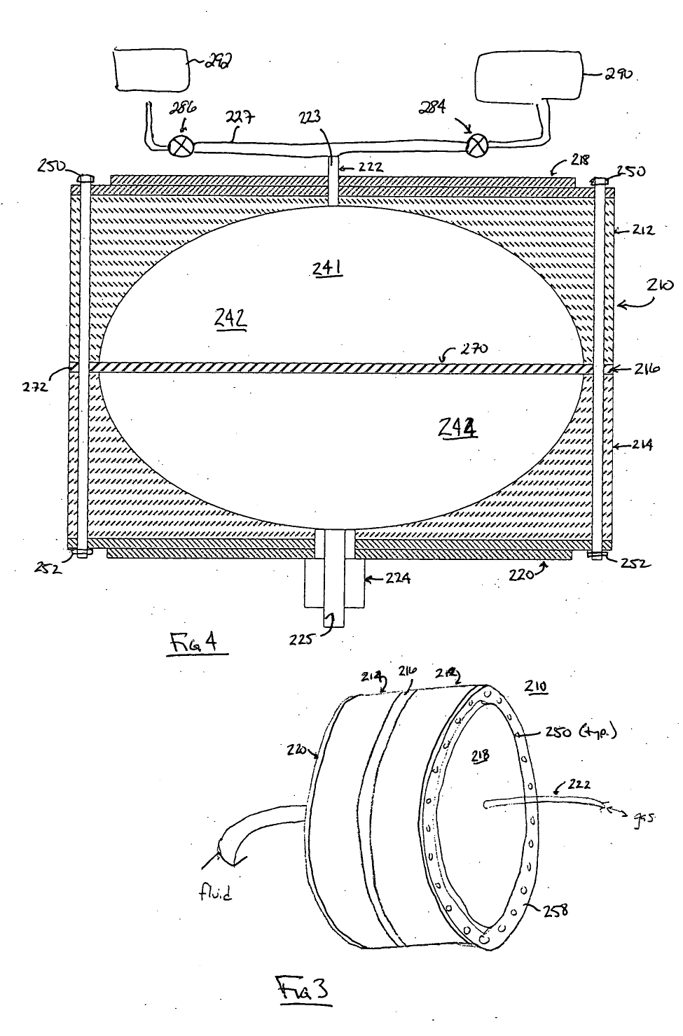

Referring now to FIG. 2, the present invention provides a multiple bladder pump system 110 which provides for a more continuous pumping cycle. Pump system 110 includes an elongate hollow fluid line 112 defining a medially-located inlet port 114 and opposed first and second outlet ports 116 and 117. Fluid line 112 further defines an elongate passageway 118 extending between inlet port 114 and outlet ports 116 and 117 for transporting hyperpolarized gases therethrough. First and second inlet valves 120 and 121 and first and second outlet valves 122 and 123 are spacedly positioned in fluid communication with passageway 118 of fluid line 112 so as to provide interruptible and directable flow within and through fluid line 112. Pump system 110 includes a first pump chamber 124 defining an interior cavity 126. A first collapsible gas bladder 128 is affixed within cavity 126 so as to define a gas cavity 130 in fluid communication with passageway 118 of fluid line 112 at a location between first inlet and first outlet valves 120 and 122. Gas bladder 128 includes a bladder wall 132 which further defines a first fluid cavity 134 opposite bladder wall 132 from gas cavity 130.

Pump system 110 further includes a second pump chamber 136 defining a second cavity 127. A second collapsible gas bladder 129 is affixed within cavity 127 so as to define a gas cavity 131 in fluid communication with passageway 118 of fluid line 112 at a location between second inlet and second outlet valves 121 and 123. Gas bladder 129 includes a bladder wall 133 which further defines a second fluid cavity 138 opposite bladder wall 133 from gas cavity 131.

A pumping mechanism 140 is positioned in fluid communication between first and second fluid cavities 134 and 138. Pumping mechanism 140 is able to direct drive fluid 142 between first and second fluid cavities 134 and 138 so as to simulataneously urge gas bladder 128 between an inflated and a deflated configuration and gas bladder 129 between a deflated and an inflated configuration. Pumping mechanism 140 is contemplated to be either a reversible pump, or a one-way pump and a system of valves employed to pump the fluid in either direction. Likewise, pumping mechanism 140 may be pressure-compensated, or a bypass valve may be included to allow for deadhead operation. As can be seen, first and second fluid cavities 134 and 138 further act as fluid reservoirs for pump chambers 136 and 124, respectively.

Pump system 110 desirably employs as drive fluid 142 an incompressible fluid within fluid cavities 134 and 138 and within pumping mechanism 140, although it is also contemplated that drive fluid 142 may be a compressible fluid or gas as well. Actuation of pumping mechanism 140 causes the transfer of fluid 142 between fluid cavities 134 and 138 so as to cause the sequential or intermittent inflation and deflation of gas bladders 128 and 129. Inlet and outlet valves 120-123 are operably associated with the inflation and deflation of gas bladders 128 and 129 so as to alternately direct the flow of hyperpolarized gas from inlet port 112 into gas cavities 130 and 131 and through outlet ports 116 and 117 as will be described further hereinbelow. Pump system 110 desirably includes pressure guages 150 and 152 in pressure communication with first and second fluid cavities 134 and 138, respectively, for monitoring system pressure without requiring direct measurement of the gas pressure within the hyperpolarized gas-conducting passgeway.

The use of an incompressible hydraulic liquid around the flexible gas bladders material is expected to reduce the amount of gas contaminants that permeate the bag. However, the pumping systems of the present invention desirably employ hydraulic pumping, each pumping system could also work pneumatically, using a compressed gas cylinder or air compressor in place of the hydraulic system

The pumping cycle for pump system 110 may be as follows:

1) Inlet valve 120 and outlet valve 123 are closed, fluid 142 is pumped from second fluid cavity 138 to first fluid cavity 134, and inlet valve 121 and outlet valve 122 are opened. During this step, hyperpolarized gas is drawn into gas bladder 129 through inlet 114 while being forced out of gas bladder 128 through outlet port 116.

2) Inlet valve 121 and outlet valves 122 are closed, fluid 142 is pumped from first fluid cavity 134 to second fluid cavity 138, and inlet valve 120 and 123 are opened. During this step, hyperpolarized gas is drawn into gas bladder 128 through inlet port 114 and forced out of gas bladder 129 through outlet port 117.

3) repeat

Operated this way, pump system 110 delivers a quasi-continuous flow of gas from inlet port 114 to outlet ports 116 and 117. It is further contemplated that additional pumps of the present invention could be added to a pump system of the present invention, or that multiple pump systems of the present invention, may be operated at staggered cycles so as to provide a still smoother and more continuous supply of gas.

The pump systems of the present invention have several important advantages over prior art gas handling systems. Pump systems 10 and 110 are expected to lose no more than about 5% of the polarization during the pumping cycle. Additionally, monitoring the pressure of fluid 42 or 142 surrounding one or both of the identified gas bladders is equivalent to monitoring the pressure of the polarized gas in the bag. Nearly all commercially available pressure gauges contain wetted parts that cause hyperpolarized gases to relax quickly, so this is a significant advantage. Furthermore, the present invention provides for relating the flow of hyperpolarized gas into or out of each gas bladder to the flow of fluid through the pumping mechanism, thereby allowing the precise metering of the hyperpolarized gas flow by metering of the fluid flow. The present invention provides this feature, once again, by avoiding the deleterious effects on polarization caused by flow meters and metering valves directly in contact with the hyperpolarized gas stream.

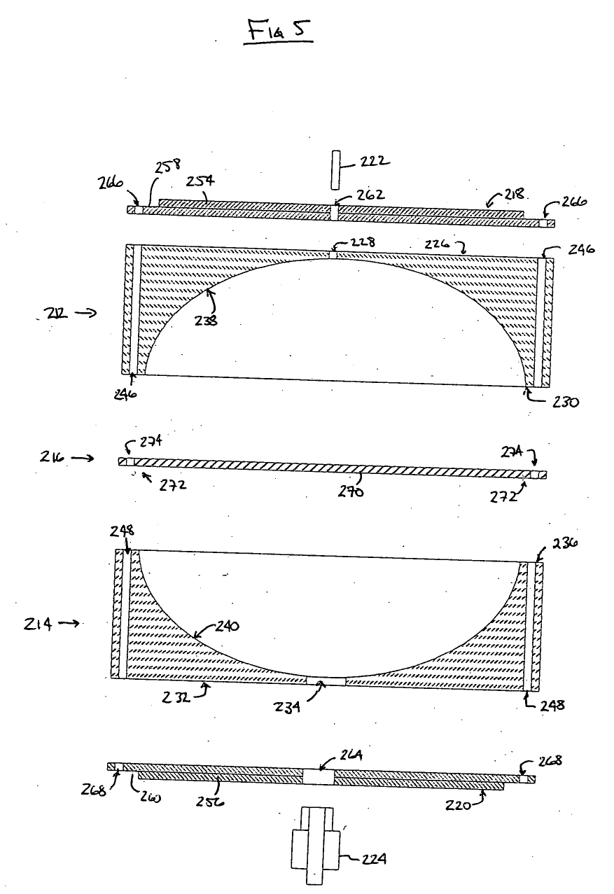

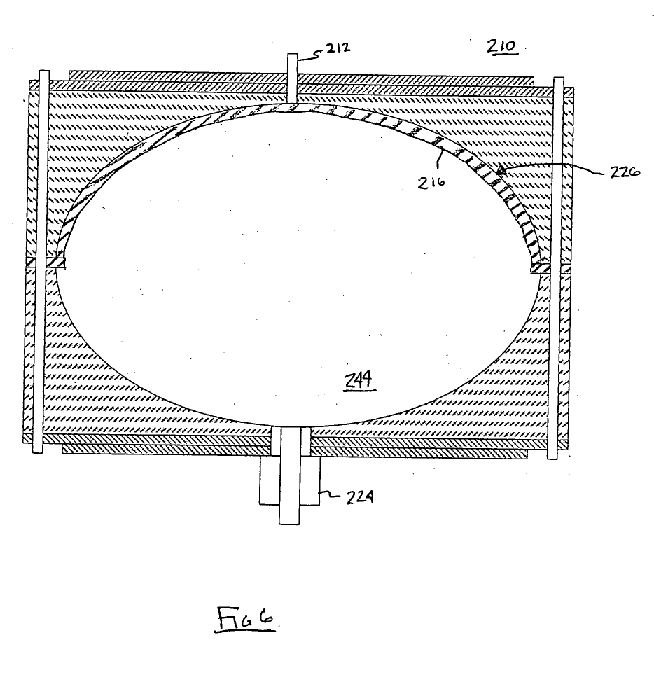

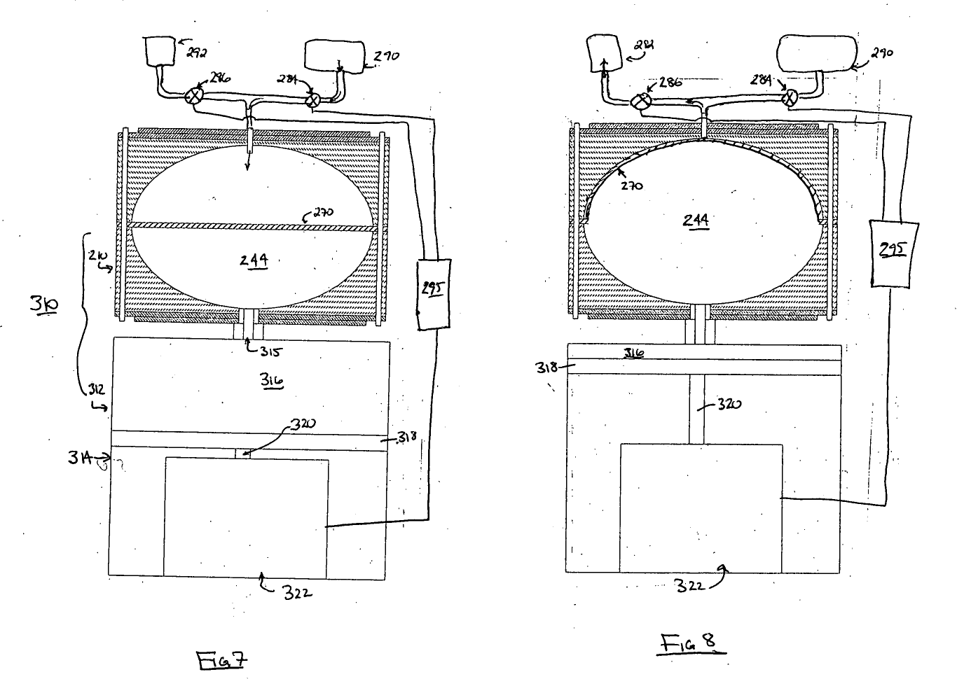

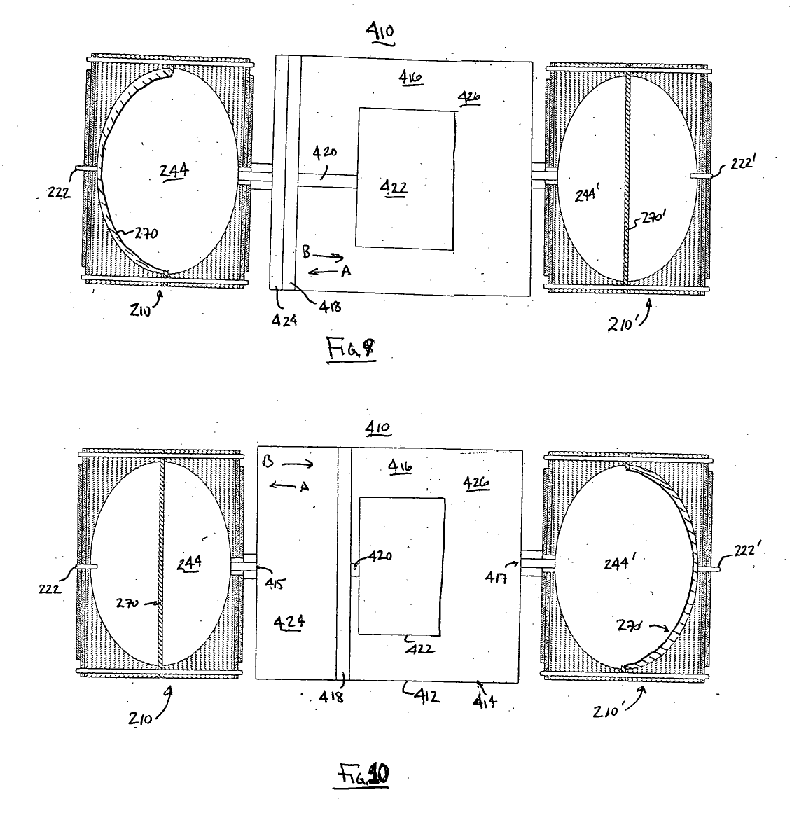

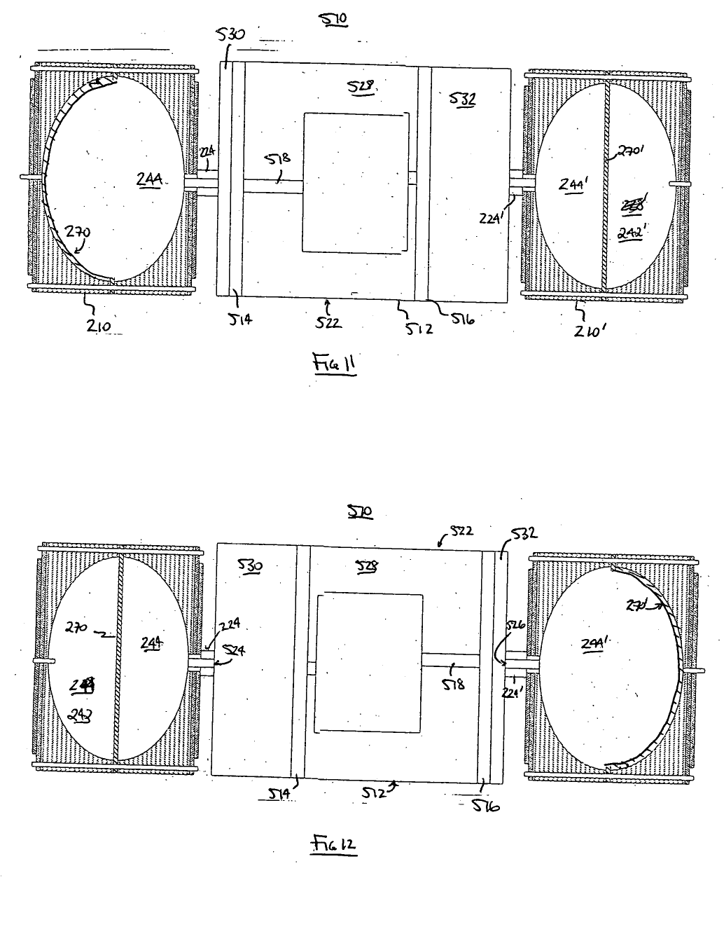

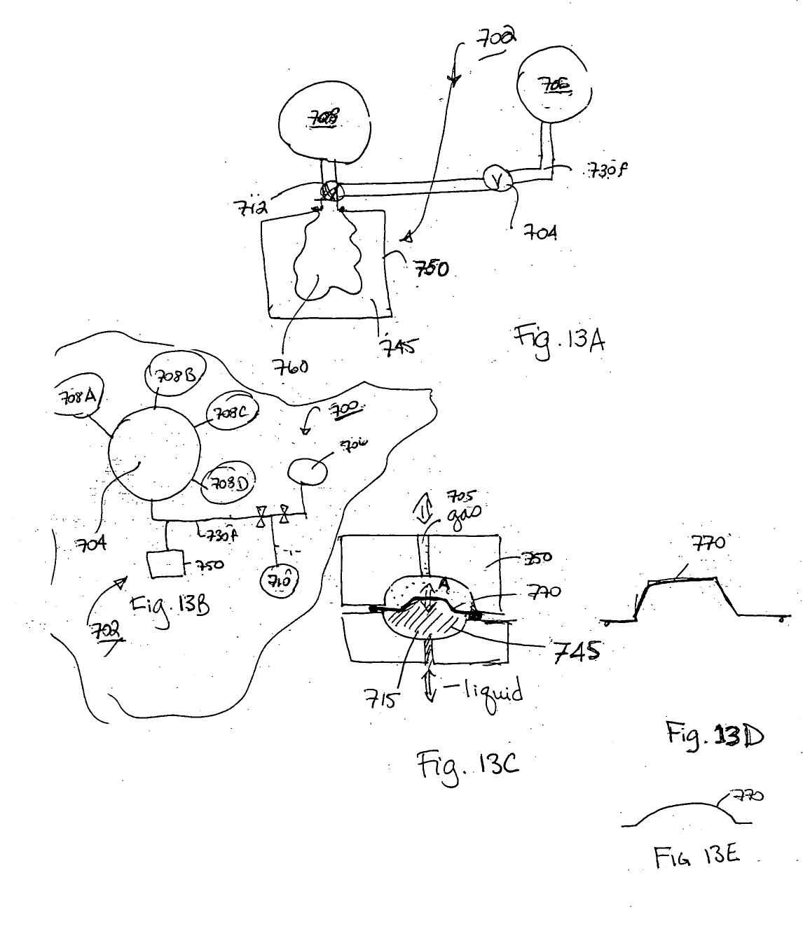

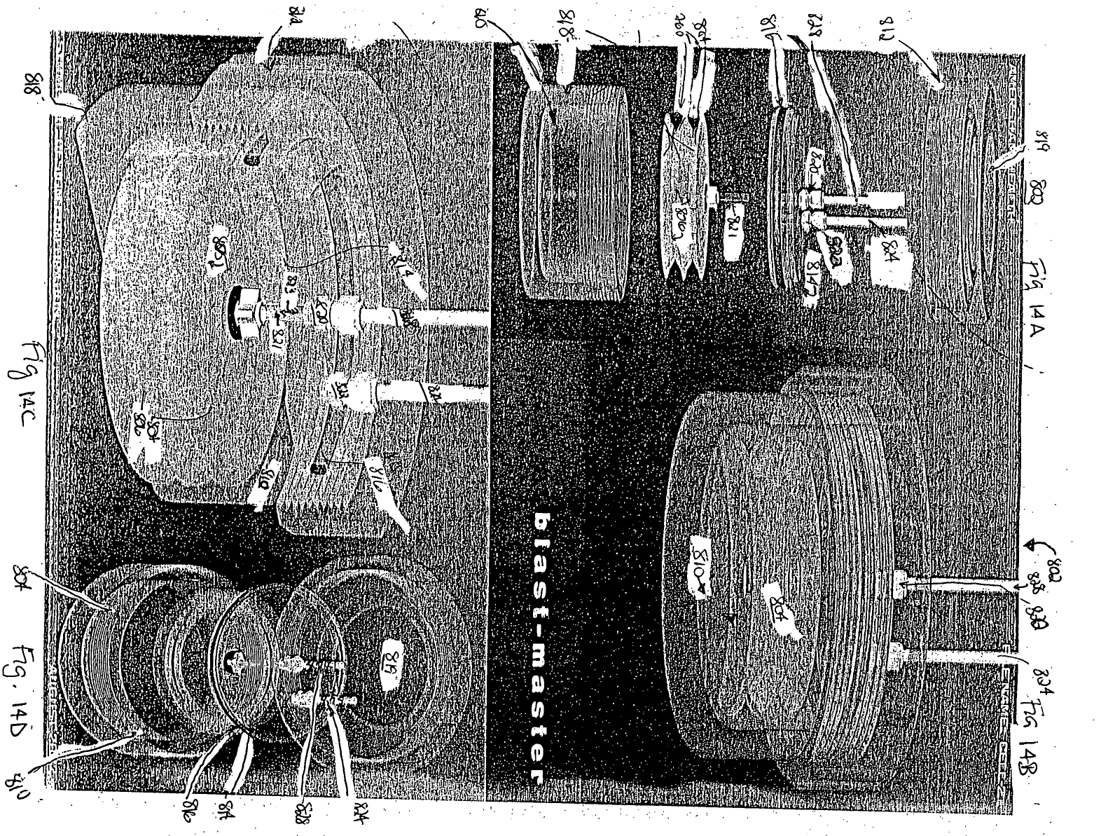

Referring now to FIGS. 3 and 4, the present invention also provides a cylindrical pump chamber 210 for a pump system for delivering hyperpolarized gas with minimal impact on the polarization level of the gas. Pump chamber 210 includes first and second housing members 212 and 214, flexible bladder 216, and endcaps 218 and 220. A fluid line 222 for conducting hyperpolarized gas to and from pump chamber 210 leads from one end of pump chamber 210 while a second fluid line or fitting 224 is provided on the opposite end of pump chamber 210. Fluid line 222 defines an elongate gas passageway 223 while fitting 224 defines an elongate fluid passageway 225 therethrough.

First and second housing members 212 and 214 are desirably formed from polycarbonate, although other polymeric materials which are known to have a low impact on gas polarization may also be employed. Endcaps 218 and 220 may be formed from aluminum or other like materials so as to impart structural rigidity to the reservoir assembly. Neither of endcaps 218 and 220 are contemplated to come into direct contact with a hyperpolarized gas. Flexible bladder 216 is desirably formed from a durable polymer material which exhibits the ability to flex while having low impact on the polarization level of a gas conducted by fluid line 222.

With additional reference to the exploded view of FIG. 5, first housing member 212 includes a planar major surface 226 defining a central aperture 228 for receiving fluid line 222. First housing member 212 also includes annular surface 230 in facing opposition to major surface 226. Similarly, second housing member 214 includes a planar major surface 232 defining a central aperture 234 for receiving fitting 224. Second housing member 214 further includes annular surface 236 in facing opposition to major surface 232. First housing member 212 includes a spherical interior surface 238 and second housing member 214 includes an opposing spherical interior surface 240 which, therebetween, defines interior cavity 241. The interposition of bladder 216 between surfaces 238 and 240 further defines gas cavity 242 opposite bladder 216 from fluid cavity 244. First and second housing members 212 and 214 further define a plurality of circumferentially-located fastener apertures 246 and 248 for accommodating bolt and nut fasteners 250 and 252 therethrough.

Endcaps 218 and 220 include circular bodies 254 and 256 and have an outer annular flange 258 and 260, respectively. Endcap 218 defines a centrally-located aperture 262 extending therethrough for accommodating fluid line 222. Endcap 220 defines a centrally-located aperture 264 extending therethrough for accommodating fitting 224. Annular flanges 258 and 260 define a plurality of fastener apertures 266 and 268, resepectively, therethrough for accommodating bolt and nut fasteners 250 and 252.

Bladder 216 has the form of a circular diaphragm 270 having a peripheral edge 272 compressed between opposed annular surfaces 230 and 236 of first and second housing members 212 and 214. Diaphragm 270 defines a plurality of through-apertures 274 circumferentially-located along peripheral edge 272 and which extend in registry with fastener apertures 246 and 248 of first and second housing members 212 and 214 so as to accommodate bolts 250 therethrough.

The present invention contemplates that a fluid may be delivered into and out of fluid cavity 234 so as to cause bladder 216 to deflect alternately into gas cavity 242, so as to force out the hyperpolarized gas therein, and into fluid cavity 244 so as to draw hyperpolarized gas from fluid line 222. As shown in FIG. 3, fluid line 222 is in fluid communication with elongate fluid line 227 having valves 284 and 286 at opposed ends thereof so as to control the directional flow of hyperpolarized gas from its source 290 to either a patient or a storage container 292. FIG. 6 depicts bladder 216 deflected against interior surface 226 so as to fully expel any hyperpolarized gas from gas cavity 228. By describing interior surfaces as ‘spherical’, the present invention also contemplates that the surface desirably exhibits any shape against which bladder 216 is able to conform when deflected.

Referring now to FIGS. 7 and 8, the present invention provides a pump system 310 for hyperpolarized gas which incorporates pump chamber 210. Pump system 310 includes a fluid transfer device 312 including a housing 314. Fluid housing 314 defines a fluid aperture 315 and a fluid drive chamber 316 in fluid communication with fluid cavity 244. Fluid transfer device 312 also includes a piston drive mechanism including a piston head 318 affixed to one end of an elongate piston rod 320. A moveable piston actuator 322 urges piston head towards and away from fluid aperture 315 so as to cause the deflection of diaphragm 270 and the controlled delivery of hyperpolarized gas into and out of gas cavity 242. An electronic controller 295 includes the necessary circuits and software for coordinating the opening and closing of valves 284 and 286 with the operation of piston actuator 322. Controller 295 opens valve 284, closes valve 286, and causes piston head 318 to retract towards actuator 322. This action causes the flow of fluid from fluid cavity 244 into drive chamber 316, which draws diaphragm 270 towards surface 240, causing hyperpolarized gas to flow from a source 290 into gas cavity 242. Then, controller 295 closes valve 284, opens valve 286, and causes piston head 318 to extend towards aperture 315. This action causes the flow of fluid into fluid cavity 244 from drive chamber 316, which extends diaphragm 270 towards surface 238, causing hyperpolarized gas to flow into storage container 292 from gas cavity 242.

FIGS. 9 and 10 depict another pump system 410 of the present invention. Pump system 410 incorporates first and second pump chambers 210 and 210′ of the present invention and a fluid transfer device 412 which causes the reservoirs to alternately draw in and expel hyperpolarized gas. Fluid transfer device 412 includes a housing 414 which defines first and second fluid apertures 415 and 417. Housing 414 defines an interior cavity 416 and includes a single piston head 418 affixed to one end of an elongate piston rod 420 which is urgable in the directions of arrows A and B by an acutator 422. Piston Head 418 divides cavity 416 into a first drive chamber 424 in fluid communication with aperture 415 and fluid cavity 244 and a second drive chamber 426 in fluid communication with aperture 417 and fluid cavity 244′. Fluid drive chambers 424 and 426 are filled with the same fluid as fluid cavities 244 and 244′, respectively. Movement of piston head 418 in each direction causes diaphragms 270 and 270′ to simultaneously, but alternately, deflect between their first and second positions as shown in FIGS. 9 and 10. Pump system 410 is thereby able to provide more continuous flow of hyperpolarized gas to through fluid conduits 222 and 222′ which are desirably connected to a common destination although separate destinations are also contemplated.

FIGS. 11 and 12 depict another pump 510 of the present invention. Pump 510 is similar to pump 410 in that it incorporates a first and second reservoir 210 and 210′ with a fluid transfer device 512. Fluid transfer device 512 includes first and second piston heads 514 and 516 affixed to opposing ends of elongate piston rod 518. Fluid transfer device 512 includes a housing 522 which defines apertures 524 and 526 for accommodating fittings 224 and 224′, respectively. Housing 522 further defines an interior cavity 528. First piston head 514 and housing 522 further define first drive chamber 530 which is in fluid communication with aperture 524 and first fluid cavity 244 of reservoir 210. Second piston head 516 and housing 522 further define second drive chamber 532 which is in fluid communication with aperture 526 and second fluid cavity 244′ of reservoir 210′. Piston actuator 520 controls movement of piston heads 514 and 516 in the directions of arrows A and B so as to cause the simultaneous, but alternating, deflection of diaphragms 270 and 270′ which thereby provides more continuous flow of a hyperpolarized gas from gas cavities 242 and 242′.

FIG. 13A illustrates a gas distribution system 700 having selectable flow paths 730f with a gas transfer mechanism 702 that uses pressure differentials to direct target gas between the optical pumping cell 720 and the selected holding cell 706. As shown, the gas distribution valve 704 is in fluid communication with the holding cells 706 (shown for clarity as a single cell) and the polarization or optical pumping cell 708. FIG. 13B illustrates that the gas distribution system 700 uses the gas distribution valve 704 to serially connect or a desired holding cell 706 and connect it to the gas transfer mechanism 702 so as to be able to flow the target gas in a desired direction.

FIG. 13B shows the valve 704 as it connects to each holding cell 708A-708D in a gas distribution system 700 that uses the gas transfer mechanism 702 to direct the target gas to and from the optical pumping cell 706 as well as to mete out or deliver doses of the polarized gas to the dispensing port 710. Referring again to FIG. 13A, the gas transfer mechanism 702 employs a housing with a pressure chamber 750 and a resilient or compressible member 760. In the embodiment shown, the resilient member 760 is an elastomeric bag, such as a TEDLAR bag, or other bag formed of or coated with materials that can provide a suitable T1 for polarized gas. Valve 712 is optional and may be a glass valve used to isolate the holding cell and/or transfer mechanism 702. In operation, fluid, typically an incompressible liquid such as oil (which may be a non-toxic biodegradeable oil) is directed into the cavity 745 of pressure chamber 750. Alternatively, a compressible gas such as nitrogen gas may also be suitable. The input of fluid into cavity 745 compresses bag 760 and forces the gas in bag 760 out into the flow path (to the cell 708 or 706). In the reverse, removing the fluid from the chamber acts to evacuate the system and pull the target gas into bag 760.

The lines in the gas flow path of any of the pumps of the present invention can be formed of small I.D. tubing to reduce the dead volume in the lines of the flow path. For example, 0.03 inch PTFE tubing can be suitable to form portions of all of the flow paths. In certain embodiments, the gas transfer mechanism 702 can be used to provide meted volumes of polarized target gas 705 to the dispense port 710. Using an incompressible liquid such as oil, and knowing the volume, temperature and pressure of the liquid, the volume of the target gas dispensed may be calculated. The gas transfer mechanism 702 does not require a motorized pump to operate to transfer the polarized gas, but such a pump may be used to transfer non-polarized fluid (target gas, filler gas, purge gas, and the like).

FIG. 13C illustrates a pressure chamber 750 that employs a membrane 770 that extends across the cavity 745 as the resilient member 760. Membrane 770 divides cavity 745 into gas portion 745a for receiving and expelling gas 705 and fluid portion 745b for receiving and expelling a liquid compression fluid 715. Membrane 770 is desirably conformable to the shape of the cavity (i.e., the internal shape of housing of gas transfer mechanism 702). As liquid is forced into fluid portion 745b, membrane 770 deflects to push out the target gas 705. The membrane 770 may be sized to deflect sufficiently to contact the upper and/or lower walls of the cavity 745. The upper deflection occurs when sufficient liquid has been introduced into cavity 745b and the lower deflection occurs when the liquid has been withdrawn therefrom so as to thereby pull the membrane 770 down (as shown in the figure) or otherwise away from cavity 745a. Cavity 745 may be sized so that, at full deflection, the membrane 770 and cavity 745 can hold about 1.0 L of target gas therein. Other sizes may also be accommodated. As shown in FIGS. 13D and 13E, membrane 770 may be preshaped with a ramped projection profile or a dome-shape, respectively, to help push the target gas from cavity 750c. Other membrane shapes may also be useful.

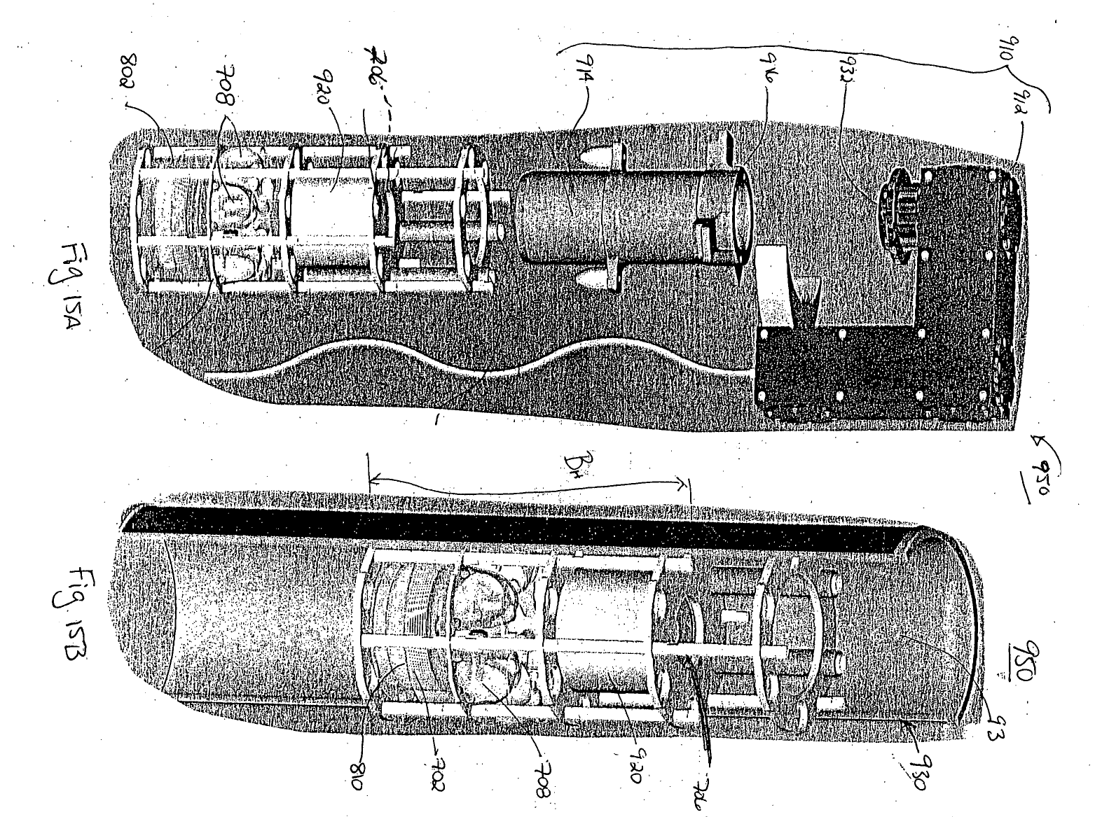

FIGS. 14A-14D illustrate a gas transfer mechanism 802 using a bladder 804 as the resilient member 760. The bladder 804 can include a series of pleats 806. The pressure chamber 810 includes a lid 812, a platform 814 supporting a seal 816, and a primary body 818. Platform 814 defines the port 820 therethrough for the target gas entry and exit as well defines the port 822 therethrough for the reserve, or driving, fluid. Lid 812 is secured to body 818 and compresses seal 816, thereby defining the pressure chamber 810. Lid 812 further defines an opening 819 for accommodating flowpaths 822 and 824 therethrough. Seal 816 is positioned in fluid-tight engagement between lid 812 and platform 814. Lid 812 and body 818 are further joined in fluid-tight engagement through any manner known to the art. Bladder 804 supports an elongate hollow stem 821 defining elongate passageway 823 in fluid communication with the interior 805 of bladder 804. Stem 821 is affixed to platform 814 so as to establish fluid communication between bladder interior 805 and flowpath 828. It is contemplated that stem 821 is received in port 820 in mating threaded engagement although the two components may be joined adhesively or in any other manner known to the art.

The pressure chamber components are sized and configured to hold the bladder 804 therein and to allow a reservoir fluid (typically an incompressible fluid or liquid) to controllably enter and exit from pressure chamber 810 via port 822 and flow path 824 (attached to the fluid source). As the reservoir fluid is delivered to, and withdrawn from, pressure chamber 810 about bladder 804, a hyperpolarized gas will be expelled from, and drawn into, bladder interior 805 via port 820 and gas flowpath 828. Controlling the valving (not shown) as described before allows gas transfer mechanism 802 to provide hyperpolarized gas to a desired location for a desired use.

FIGS. 15A-B illustrate interior components of a hyperpolarizer 950 with the gas transfer mechanism 802 positioned below the holding cells 708 and the optical pumping cell 706. One of skill in the art will readily recognize that any of the pumps of the present invention may be employed as a gas transfer mechanism in a hyperpolarizer 950. Moreover, hyperpolarizer 950 may include features described in commonly assigned U.S. Pat. No. 5,642,625, U.S. Pat. No. 6,269,648, and U.S. Provisional Patent Application No. 60/440,747, filed Jan. 17, 2003 and again filed as a U.S. Patent Application on even date herewith, the entire disclosures of which are hereby incorporated by reference as if having been fully described herein. FIG. 15A depicts a partially-exploded view of the interior components of hyperpolarizer 950. The optic system 910 includes an overhead housing 912 and optically connects to an optical tube 914 that blocks perimeter light and extends between housing 912 and the light port 922 of the oven 920 to direct the laser light to the cell 706. The optic housing 912 is suspended above a solenoid 930 (shown partially in FIG. 15B) by a bracket 932 that attaches to an upper portion 916 of the optical tube 914. Solenoid 930 provides a region of homogeneity about optical pumping cell 706, holding cells 708, and gas transfer mechanism 802. Laser radiation generated in housing 912 is directed at optical pumping cell 706.

As shown in FIG. 15B, the pressure chamber 810 of gas transfer mechanism 802, the holding cells 708 and the oven 920 with optical cell(s) 706 all extend inside the solenoid cavity 936 defined by solenoid 930 so as to extend within a region of homogeneity “BH”. The solenoid 930 may be end-compensated (with the number of coil wraps being increased on the two opposing end portions relative to the center portion of the solenoid) to increase the length of the region of homogeneity B_{H}, but typically, the region may be approximated as being in about the central third of the length of the solenoid 930. A single continuous length of 16 gauge wire (not shown) can be wrapped so as to provide a solenoid 930 with about double the number of wrappings on end portions relative to the center portion (which may have a length that is longer than the sum of the lengths of both end portions) to provide a homogenous region B_{H} that is about 8 inches in diameter and about 18 inches long. The present invention also contemplates that solenoid 930 may be formed from mu-metal so as to better shield the polarized gas.

The present invention contemplates that the pumps of the present invention are controlled by controllers which coordinate the opening and closing of the gas delivery valves with the deflection of the bladders within the reservoirs. The controllers allow for a hyperpolarized gas to be transferred from a first container to either a patient or to a secondary gas storage device. Moreover, by employing an incompressible fluid within the fluid cavities of the reservoirs, the present invention may provide for more complete withdrawal from, and delivery into, rigid containers used for storing a hyperpolarized gas. Alternatively, the pumps of the present invention may be repetitively operated to provide near-continuous flow of hyperpolarized gas to a destination, whether a patient or a storage container. The pumps of the present invention are contemplated to deflect the resilient members of the pump chambers at pressures as high as 120 p.s.i. as well as up to pressures of 200 p.s.i..

The present invention further contemplates that the actual layout of the fluid transfer device with respect to the reservoirs may be selected to accommodate housings of many varying shapes. The figures depict the desirable arrangements of these components so as to be in a substantially linear alignment, however, other spatial configurations may also be realized by the use of fluid conduits between the drive chambers of the fluid transfer device and the fluid cavities of the reservoir. It will be appreciated that the physical layout of the pump is therefore capable of most any configuration.

While the particular embodiment of the present invention has been shown and described, it will be obvious to those skilled in the art that changes and modifications may be made without departing from the teachings of the invention. The matter set forth in the foregoing description and accompanying drawings is offered by way of illustration only and not as a limitation. The actual scope of the invention is intended to be defined in the following claims when viewed in their proper perspective based on the prior art.

What is claimed is:

1. A pump for transferring hyperpolarized gases comprising:
An elongate fluid line defining an elongate passageway for transporting hyperpolarized gases therethrough;
A first inlet valve and a first outlet valve spacedly positioned in fluid communication with said fluid line so as to provide interruptible and directable flow therethrough;
A first pump reservoir defining an interior cavity, said first pump reservoir further comprising a deflectable gas bladder defining a gas cavity in fluid communication with said fluid line between said first inlet and outlet valves, said gas bladder further defining a first fluid cavity opposite said bladder from said gas cavity;
A fluid reservoir defining a second fluid cavity; and
A pumping mechanism able to direct fluid between said first and second fluid cavities so as to urge said gas bladder between a first and second configuration.

2. The pump of claim 1, wherein said first and second fluid cavities contain an incompressible fluid.

3. The pump of claim 1, wherein said first and second fluid cavities contain a compressible fluid.

4. The pump of claim 1, wherein said gas bladder is formed from a gas- impermeable material.

5. The pump of claim 1, wherein said gas bladder is formed from a material selected for having a low impact on the level of polarization of a hyperpolarized gas contained within said gas cavity.

6. The pump of claim 1, further comprising a first pressure gauge in fluid communication with one of said first and second fluid cavities.

7. The pump of claim 6, further comprising a second pressure gauge in fluid communication with the other of said first and second fluid cavities.

8. The pump of claim 1, further comprising:
A second inlet valve and a second outlet valve spacedly positioned on said fluid line so as to provide interruptible and directable flow therethrough; and
wherein said fluid reservoir further comprises a second pump chamber comprising a second deflectable gas bladder defining a gas cavity in fluid communication with said fluid line between said second inlet and outlet valves, said second gas bladder further fluidly isolating said second gas cavity from said second fluid cavity, said second inlet and outlet valves operably associated with said first inlet and outlet valves.

9. The pump of claim 8, wherein said first and second inlet valves are positioned exteriorly to said first and second outlet valves along said fluid line, said fluid line further defining a gas inlet port between said outlet valves.

10. The pump of claim 8 wherein said first and second inlet valves are positioned between said first and second outlet valves along said fluid line, said fluid line further defining a gas outlet port between said inlet valves.

11. The pump of claim 1, wherein said first pump chamber further comprises a first housing member defining said gas cavity and a second housing member defining said first fluid cavity, and wherein said gas bladder is in the form of a deflectable diaphragm positioned between said first and second housing member so as to fluidly isolate said gas cavity from said first fluid cavity.

12. The pump of claim 9, wherein said first housing member further comprises an interior surface defining said gas cavity.

13. The pump of claim 9, wherein said first housing further comprises a semi-spherical interior surface defining said gas cavity.

14. The pump of claim 10, wherein said diaphragm is extendable to substantially conform to said interior surface.

15. The pump of claim 9, wherein said second housing member includes a semi-spherical interior surface defining said first fluid cavity.

16. The pump of claim 12, wherein said diaphragm is extendable to substantially conform to said interior surface of said second housing member.

17. The pump of claim 1, wherein said pumping mechanism further comprises a piston housing defining said second fluid cavity and including a piston head movable within said second fluid cavity so as to direct fluid between said first and second fluid cavities.

18. The pump of claim 1, wherein said deflectable gas bladder further comprises a resilient bag having a bag wall defining said gas cavity and further defining a gas port in fluid communication with said fluid line.

19. The pump of claim 14, wherein said first pump chamber further comprises a first housing member defining said gas cavity and a second housing member defining said first fluid cavity, and wherein said gas bladder is in the form of a deflectable diaphragm positioned between said first and second housing member of said first fluid reservoir so as to fluidly isolate said gas cavity from said first fluid cavity.

20. A pump for transferring hyperpolarized gases comprising:
An elongate first fluid line defining an elongate passwageway for transporting hyperpolarized gases therethrough;
A first pump chamber defining a first interior cavity, said first pump chamber comprising a first deflectable gas bladder defining a first gas cavity in fluid communication with said first fluid line, said first gas bladder further defining a first fluid cavity opposite said first gas bladder from said first gas cavity, said first gas bladder deflectable from a first configuration to a second configuration within said first interior cavity;
An elongate second fluid line defining an elongate passwageway for transporting hyperpolarized gases therethrough;
A second pump chamber defining a second interior cavity, said second pump chamber comprising a second deflectable gas bladder defining a second gas cavity in fluid communication with said second fluid line, said second gas bladder further defining a second fluid cavity opposite said second gas bladder from said second gas cavity, said second gas bladder deflectable from a first configuration to a second configuration within said second interior cavity; and
A fluid transfer device having a housing defining a first drive chamber in fluid communication with said first fluid cavity and a second drive chamber in fluid communication with said second fluid cavity, said fluid transfer device including a fluid drive mechanism for directing fluid between said first fluid cavity and said first drive chamber and between said second fluid cavity and said second drive chamber so as to respectively cause said first and second bladders to deflect between said first and second configurations.

21. The pump of claim 20, wherein said fluid drive mechanism comprises a first piston surface in facing communication with said first drive chamber and a second piston surface in facing communication with said second drive chamber.

22. The pump of claim 21, further comprising a piston head urgeable between a first position directing fluid from said first drive chamber into said first fluid cavity and a second position drawing fluid from said first fluid cavity into said first drive chamber.

23. The pump of claim 22, wherein said piston head further comprises said first and second piston surfaces.

24. The pump of claim 22, further comprising a second piston head urgeable between a first position directing fluid from said second drive chamber into said second fluid cavity and a second position drawing fluid from said second fluid cavity into said second drive chamber.

25. The pump of claim 24, wherein said first piston head further comprises said first piston surface and said second piston head further comprises said second piston surface.

26. The pump of claim 25, further comprising a piston rod extending between said first and second piston heads.

27. The pump of claim 20, wherein said first pump chamber further comprises a first housing member defining said gas cavity and a second housing member defining said first fluid cavity, and wherein said gas bladder comprises a deflectable diaphragm positioned between said first and second housing member of said first fluid reservoir so as to fluidly isolate said gas cavity from said first fluid cavity.

28. The pump of claim 27, wherein said second pump chamber further comprises a first housing member defining said second gas cavity and a second housing member defining said second fluid cavity, and wherein said gas bladder comprises a deflectable diaphragm positioned between said first and second housing member of said second fluid reservoir so as to fluidly isolate said second gas cavity from said second fluid cavity.

29. The pump of claim 27, wherein said first housing member further comprises an interior surface defining said gas cavity.

30. The pump of claim 29, wherein said first housing further comprises a semi-spherical interior surface defining said gas cavity.

31. The pump of claim 30, wherein said diaphragm is extendable to substantially conform to said interior surface.

32. The pump of claim 28, wherein said second housing member includes a semi-spherical interior surface defining said first fluid cavity.

33. The pump of claim 32, wherein said diaphragm is extendable to substantially conform to said interior surface of said second housing member.

34. The pump of claim 28, wherein said first reservoir, said second reservoir, and said fluid drive mechanism are in substantially linear alignment.

35. The pump of claim 20, further comprising a pressure gage measuring fluid pressure in said first fluid cavity.

36. The pump of claim 20, wherein said fluid transfer device can alternately direct enough fluid into each of said first and second fluid cavities so as to fully expel any hyperpolarized gas from at least one of said first and second gas cavities, respectively.
